(19) United States
(12) Patent Application Publication
(10) Pub. No.: US 2006/0116636 A1
(43) Pub. Date: Jun. 1, 2006

(54) Self-sealing catheter for deformable tissue

(76) Inventors: Kieran P. Murphy, Baltimore, MD (US); Michael Williams, Baltimore, MD (US); Daniele Rigamonti, Baltimore, MD (US)

Correspondence Address: TORYS LLP, 79 WELLINGTON ST. WEST, SUITE 3000, TORONTO, ON, M5K 1N2, CA

(21) Appl. No.: 10/998,676

(22) Filed: Nov. 30, 2004

(51) Int. Cl. A61M 29/00

(52) U.S. Cl. 604/104; 604/530

(57) ABSTRACT

A self-sealing catheter for use in non-conformable tissue is provided. In an embodiment, the catheter includes a tube with an expandable portion that comprises nitinol. Upon an increase in temperature, the expandable portion of the tube expands to conform to the surrounding non-conformable tissue such that the expandable portion in the expanded state provides a seal between an exterior of the catheter and the non-conformable tissue. In a specific embodiment, the nitinol is embedded in the wall of the expandable portion of the tube in a braided mesh configuration, and provides a radial force that substantially prevents the expandable portion of the tube from collapsing when bent. Additionally, the catheter can include one or more radiopaque markers to assist in X-ray guided placement of the catheter in a patient's tissue. In another embodiment, the catheter includes an expandable coating surrounding the expandable portion of the tube, wherein the expandable coating comprises a hydrogel polymer. Upon an increase in temperature, the expandable coating expands to conform to the surrounding non-conformable tissue such that the expandable coating in the expanded state provides a seal between an exterior of the catheter and the non-conformable tissue.

### FIELD OF THE INVENTION

The present invention relates generally to catheters and more particularly relates to a catheter with an expandable portion for insertion into a non-conformable tissue.

### BACKGROUND OF THE INVENTION

Catheter insertion can be used for cerebrospinal fluid (“CSF”) pressure monitoring and controlled CSF drainage for the diagnosis of adult hydrocephalus (see for example, discussions in Williams M A, Razumovsky A Y, Hanley D F. Comparison of Pcsf monitoring and controlled CSF drainage to diagnose normal pressure hydrocephalus. Acta Neurochir 1998, 71: 328-330; and Haan J, Thomeer R T. Predictive value of temporary external lumbar drainage in normal pressure hydrocephalus. Neurosurgery 1988, 22: 388-391; the contents of which are incorporated herein by reference.) In addition, catheter insertion can be used for brain surgery (see for example, Naff N J, Carhuapoma J R, Williams M A, Bhardwaj A, Ulatowski J A, Bederson J, Bullock R, Schmutzhard E, Pfausler B, Keyl P M, Tuhrim S, Hanley D F. Treatment of intraventricular hemorrhage with urokinase: effects on 30-Day survival. Stroke 2000, April, 31(4): 841-7; the contents of which are incorporated herein by reference.).

Surgical and other medical procedures are often performed at sites within a patient's body where the tissue is non-conformable. Examples of non-conformable tissue are the meninges (i.e. membranes covering the brain and the spinal cord such as the dura mater) and the brain, where the tissue is deformable but does not conform to the shape of any object that may touch or pierce it. In the past, when a surgical procedure was performed on such non-conformable tissue, there would be a gap or lack of a seal between the surgical instrument and the non-conformable tissue causing a leakage of bodily fluids from the opening in the tissue. In the case of surgery involving a spinal catheter insertion, leakage of CSF between the dura mater and the spinal catheter has been known to cause inaccurate CSF pressure measurements, uncontrolled CSF drainage, and other complications. Further, in the case of brain surgery, leakage of blood and other bodily fluids between the brain and the catheter, and/or reflux of an infused drug (e.g. a clot dissolving agent such as Tissue Plasminogen Activator) along the catheter tract, has been known to cause excessive bleeding from the brain and consequential death of the patient.

Certain catheters and catheter insertion techniques do not adequately address the problem of leakage of body fluids from the opening between the catheter and the non-conformable tissue.

### SUMMARY OF THE INVENTION

It is an object of the invention to provide a novel catheter for use in non-conformable tissue that obviates or mitigates at least one of the above-identified disadvantages of the prior art.

A first aspect of the invention provides a catheter for use in a non-conformable tissue. The catheter comprises a tube for insertion into the non-conformable tissue. The tube comprises a first portion and a second portion. The first portion is expandable from a non-expanded state, which permits insertion of the catheter into the non-conformable tissue, to an expanded state, which is conformable to the non-conformable tissue such that the first portion of the tube in the expanded state provides a seal between an exterior of said catheter and the non-conformable tissue.

In a particular implementation of the first aspect, the first portion of the tube is expanded by an increase in temperature.

In a particular implementation of the first aspect, the first portion of the tube expands to the expanded state at temperatures above about 30° C.

In a particular implementation of the first aspect, the first portion of the tube expands to the expanded state at a temperature between about 30° C. and about 45° C.

In a particular implementation of the first aspect, the first portion of the tube expands to the expanded state at a temperature of about 37.5° C.

In a particular implementation of the first aspect, the first portion of the tube remains in the non-expanded state at temperatures below about 30° C.

In a particular implementation of the first aspect, the first portion of the tube remains in the non-expanded state at temperatures below about 25° C.

In a particular implementation of the first aspect, the first portion of the tube comprises nitinol.

In a particular implementation of the first aspect, the nitinol is embedded in an outside wall of the first portion of the tube in a configuration selected from the group consisting of a braided mesh layer of one or more wires, a cable of one or more wires, a multi-looped helix of one or more wires, and a toroid.

In a particular implementation of the first aspect, the nitinol provides a radial force that substantially prevents the first portion of the tube from collapsing when bent.

In a particular implementation of the first aspect, the first portion of the tube is between about 5 cm and about 15 cm in length.

In a particular implementation of the first aspect, the first portion of the tube is about 10 cm in length.

In a particular implementation of the first aspect, the second portion of the tube is flexible.

In a particular implementation of the first aspect, the second portion of the tube is between about 5 cm and about 15 cm in length.

In a particular implementation of the first aspect, the second portion of the tube is about 10 cm in length.

In a particular implementation of the first aspect, the tube further comprises at least one radiopaque marker.

In a particular implementation of the first aspect, the at least one radio-opaque marker is located at at least one end of the first portion, and at the distal end of the second portion.

In a particular implementation of the first aspect, the radiopaque marker includes a material selected from the group consisting of barium sulfate, gold, iodine, ionic and non ionic iodinated compounds, ethiodol, and lipiodol, tungsten, tantalum, gadolinium nitinol, silver, and combinations thereof.

In a particular implementation of the first aspect, the first portion of the tube further comprises an expandable coating surrounding the tube, the expandable coating being expandable from a non-expanded state, which permits insertion of the catheter into the non-conformable tissue, to an expanded state, which is conformable to the non-conformable tissue such that the expandable coating in the expanded state provides a seal between an exterior of the catheter and the non-conformable tissue.

In a particular implementation of the first aspect, the expandable coating is expanded by an increase in temperature.

In a particular implementation of the first aspect, the expandable coating expands to the expanded state at temperatures above about 30° C.

In a particular implementation of the first aspect, the expandable coating expands to the expanded state at a temperature between about 30° C. and about 45° C.

In a particular implementation of the first aspect, the expandable coating expands to the expanded state at a temperature of about 37.5° C.

In a particular implementation of the first aspect, the expandable coating remains in the non-expanded state at temperatures below about 30° C.

In a particular implementation of the first aspect, the expandable coating remains in the non-expanded state at a temperature below about 25° C.

In a particular implementation of the first aspect, the expandable coating comprises a hydrogel polymer.

In a particular implementation of the first aspect, the tube is tapered.

In a particular implementation of the first aspect, at least a portion of the catheter has an antibiotic coating.

In a particular implementation of the first aspect, at least a portion of the catheter has an adhesion resistant coating.

In a particular implementation of the first aspect, the non-conformable tissue is a dura mater.

In a particular implementation of the first aspect, the non-conformable tissue is a brain.

In a second aspect of the invention, there is provided a catheter for use in a non-conformable tissue. The catheter comprises a tube for insertion into the non-conformable tissue; and an expandable coating surrounding the tube. The expandable coating is expandable from a non-expanded state, which permits insertion of the catheter into the non-conformable tissue, to an expanded state, which is conformable to the non-conformable tissue such that the expandable coating in the expanded state provides a seal between an exterior of the catheter and the non-conformable tissue.

In a particular implementation of the second aspect, the expandable coating is expanded by an increase in temperature.

In a particular implementation of the second aspect, the expandable coating comprises a hydrogel polymer.

In a particular implementation of the second aspect, the tube further comprises a first portion and a second portion, said first portion being expandable from a non-expanded state, which permits insertion of the catheter into the non-conformable tissue, to an expanded state, which is conformable to the non-conformable tissue such that the first portion of the tube in the expanded state provides a seal between an exterior of said catheter and the non-conformable tissue.

In a particular implementation of the second aspect, the first portion of the tube is expanded by an increase in temperature.

In a particular implementation of the second aspect, the first portion of the tube comprises nitinol.

### BRIEF DESCRIPTION OF THE DRAWINGS

Embodiments of the present invention will now be explained, by way of example only, with reference to the attached Figures in which:

FIG. 1 is a side perspective view of a first embodiment of a catheter in accordance with the present invention, showing a tube portion with a needle placed therein, both inserted through non-conformable tissue.

FIG. 2 is a side perspective view of the catheter shown in FIG. 1, with the needle being withdrawn from inside the tube portion.

FIG. 3 is a side view of the catheter shown in FIG. 1, showing the tube portion of the catheter with an expandable portion, a tip portion, and radiopaque markers.

FIG. 4 is a side perspective view of the catheter shown in FIG. 1 being inserted through non-conformable tissue.

FIG. 5 is a side perspective view of non-conformable tissue with an opening.

FIG. 6 is a cross-sectional view of the catheter shown in FIG. 1, with the tube portion inserted into non-conformable tissue, wherein the expandable portion of the tube portion is expanding from a non-expanded state.

FIG. 7 is a cross-sectional view of the catheter shown in FIG. 1, with the tube portion inserted into non-conformable tissue, wherein the expandable portion of the tube portion is in an expanded state.

FIG. 8 is a side view of the catheter shown in FIG. 1, with the expandable portion of the tube portion in a non-expanded state.

FIG. 9 is a side view of the catheter shown in FIG. 1, with the expandable portion of the tube portion in an expanded state

FIG. 10 is a side view of the catheter shown in FIG. 1, showing the braided mesh configuration of the expandable material embedded in the wall of the expandable portion of the tube portion.

FIG. 11 is a side perspective view of the catheter shown in FIG. 10 with the expandable portion of the tube portion in a bent position and in an expanded state.

FIG. 12 is a partial perspective view of another embodiment of a catheter in accordance with the present invention, showing a toroidal shaped expandable material in a helix configuration embedded in the wall of the tube portion.

FIG. 13 is a top perspective view of the expandable material shown in FIG. 12.

FIG. 14 is a partial perspective view of the catheter shown in FIG. 12, showing the tube portion inserted through non-conformable tissue, with the toroidal shaped expandable material in a non-expanded state.

FIG. 15 is a partial perspective view of the catheter of FIG. 14 with the toroidal shaped expandable material in an expanded state.

FIG. 16 is a cross-sectional view of the catheter of FIG. 14 with the toroidal shaped expandable material in an expanded state.

FIG. 17 is a partial side-sectional view of another embodiment of a catheter in accordance with the present invention, showing an expandable coating covering the tube portion, wherein the tube portion is inserted through non-conformable tissue, and the expandable coating is in a non-expanded state.

FIG. 18 is a partial side-sectional view of the catheter of FIG. 17 with the expandable coating in an expanded state.

FIG. 19 is a perspective view of a catheter in accordance with an embodiment of the invention inserted between vertebrae.

FIG. 20 is top cross-sectional view of the catheter of FIG. 19 inserted through the dura mater and into the cerebrospinal canal.

FIG. 21 is a top sectional view of a catheter of FIG. 19 inserted through a previously drilled hole in a skull.

### DETAILED DESCRIPTION OF THE INVENTION

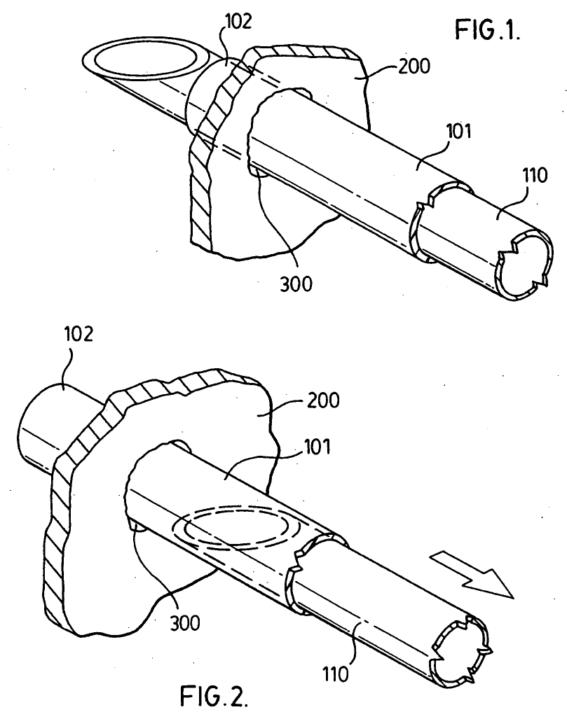

Referring to FIGS. 1-3, an embodiment of the invention includes a catheter 100 for insertion through a patient's tissue 200. Catheter 100 comprises an elongated tube 101 of a substantially tubular configuration, having a central lumen 120, a tip portion 140 at the distal end, and an expandable portion 160 located proximal from tip portion 140. In addition, catheter 100 has a removable needle 110. Tube 101 of catheter 100 is open at the distal end 102, and needle 110 is able to travel through catheter 100 and extend out from catheter's distal end 102 in order to pierce the patient's tissue 200. Once catheter 100 has been inserted into the patient's tissue 200 and to the desired site, needle 110 can be removed by withdrawing it from catheter 100. In another embodiment, tube 101 is tapered at the distal end. As will occur to those of skill in the art, other means of piercing the patient's tissue 200 can be used as the needle 110. For example, needle 110 can be a micropuncture guidewire or a Touhy needle.

Tube 101 is made from a material that is substantially rigid enough to effect the desired piercing through a patient's tissue 200, but is also substantially flexible enough to effect the desired bending and traveling through patient's tissue 200. In one embodiment, tip portion 140 of tube 101 is more flexible and softer than the expandable portion 160. The flexibility and softness of the tip portion 140 is desirable because it is substantially safer for the patient by reducing tissue damage when catheter 100 is navigated through the patient's tissue 200. Tube 101 can be made of a material that is biocompatible, adhesion-resistant, infection-resistant, flexible, elastic and/or conformable. Presently preferred materials for tube 101 include silicon and plastics such as polyethylene PTFE nylon.

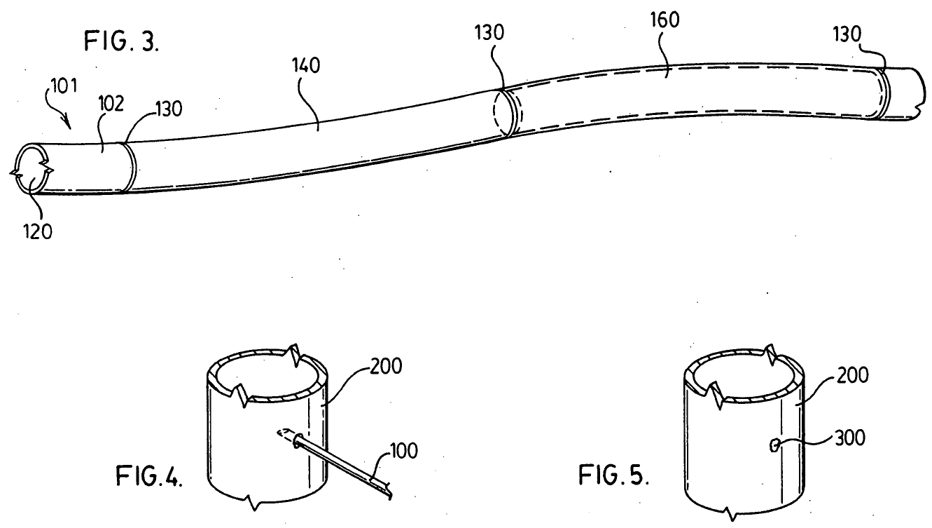

Referring to FIG. 3, it is presently preferred that tube 101 of catheter 100 include one or more radiopaque markers 130 to improve radiopacity and assist in the positioning and locatability of portions of catheter 100 during surgical procedures. The radiopaque markers 130 allow radiographic identification of one or more locations of interest on catheter 100 under an X-ray or other conventional imaging techniques (under which catheter is intended for use), and are intended to assist in X-ray guided placement of catheter 100 in patient's tissue 200.

Presently preferred locations of radiopaque markers 130 include the distal tip 102 of the tip portion 140 of tube 101, and both ends of the expandable portion 160. It is to be understood that radiopaque markers 130 can be positioned at a location on or near the expandable portion 160, or at one or both of the ends of the expandable portion 160, such that radiopaque markers 130 indicate when the expandable portion 160 is straddling a patient's dura mater 201 during surgical procedures.

Presently preferred radiopaque markers 130 include water insoluble materials such as barium sulfate, gold, iodine, ionic and non ionic iodinated compounds, ethiodol, and lipiodol, tungsten, tantalum, gadolinium nitinol, silver, or combinations thereof.

The length of the tip portion 140 of tube 101 is presently preferred to be from about 5 cm to about 15 cm in length, and more presently preferred to be about 10 cm in length. The length of the expandable portion 160 of tube 101 is presently preferred to be from about 5 cm to about 15 cm in length, and more presently preferred to be about 10 cm in length.

Referring to FIGS. 4 and 5, catheter 100 can be inserted through a patient's tissue 200 that is substantially non-elastic and non-comformable, such that an opening 300 remains between catheter 100 and tissue 200 after insertion of catheter 100. Such non-conformable tissue 200 is not self-sealing and opening 300 remains in it after catheter 100 is removed.

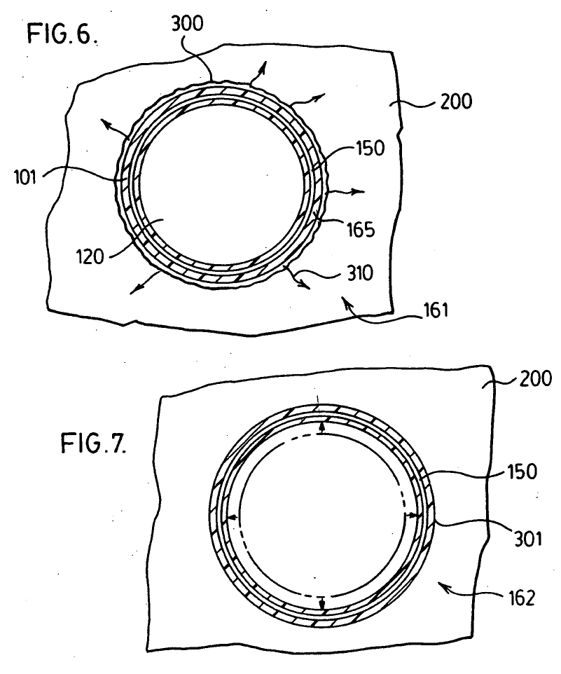

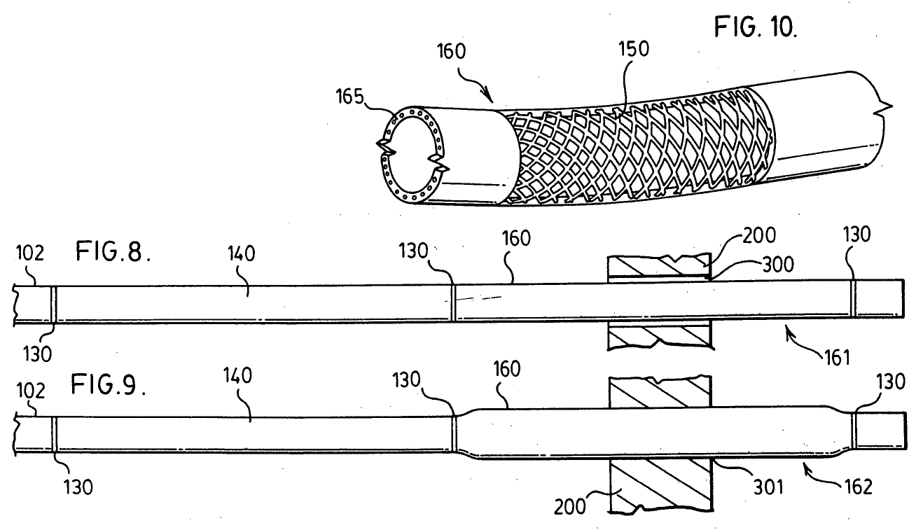

Referring to FIGS. 6-9, tube 101 comprises an expandable material 150. The expandable material 150 can be embedded within wall 165 of tube 101. When expandable portion 160 of tube 101 is expanded to an expanded state 162, it is conformable to the surrounding non-conformable tissue 200 to provide a substantially fluid-tight seal 301 between the exterior of tube 101 and the non-conformable tissue 200. As such, the opening 300 between catheter 100 and the non-conformable tissue 200 is closed by the radial expansion 310 of the expandable portion 160 out towards the tissue 200. A presently preferred expandable material 150 is nitinol. Leakage of CSF during surgery with catheter 100 is reduced and/or substantially prevented by the seal 301.

The expandable material 150 remains in a non-expanded state 161 at temperatures below a “non-expansion temperature”, which in a presently preferred embodiment, the expandable material is operable to expand to an expanded state 162 at an “expansion temperature” (e.g. a patient's body temperature) that is above the non-expansion temperature. The non-expansion temperature is presently preferred to be from about 20° C. to about 30° C., and more presently preferred to be about 25° C. The expansion temperature is presently preferred to be from about 30° C. to about 45° C., and more presently preferred to be about 37.5° C. The expansion temperature is presently preferred to approximate a patient's body temperature range.

The expandable material 150 is presently preferred to be nitinol (a nickel/titanium alloy). Examples of nitinol are described in U.S. Pat. No. 6,706,053 to Boylan et al.; and in Ponec D, Jaff M R, Swischuk J, Feiring A, Laird J, Mehra M, Popma J J, Donohoe D, Firth B, Keim E, Snead D; CRISP Study Investigators, The Nitinol SMART stent vs Wallstent for suboptimal iliac artery angioplasty: CRISP-US Trial results. J Vasc Interv Radiol. 2004 September;15(9):911-8; the disclosures of which are incorporated herein by reference.

It is presently preferred that the expandable portion 160 have an initial thickness of not more than about 4 mm; and with a thickness in the expanded state 162 of at least about 6 mm. It is also presently preferred that the expandable material 150 of the expandable portion 160 expands to a diameter of at least about 2 times from the diameter of its initial non-expanded state, more presently preferred to be at least about 1.5 times from the diameter of its non-expanded state, and still more presently preferred to be about 1.25 times from the diameter of its non-expanded state.

In addition to being expandable, expandable material 150 is also flexible, and has an elastic memory whereby it can be formed into a desired shape to which it can return when it is deformed. As will occur to those of skill in the art, the expandable material 150 can be in a variety of configurations, such as a braided mesh of one or more wires, a cable of one or more wires, a helix of one or more wires, a layer (i.e. sheet-like expanse), a toroid (i.e. ring-shaped configuration), or with geometries such as a coil design, a helical spiral design, a woven or braided design, a ring design, a sequential ring design, a closed cell design, or an open cell design, as described in D. Stoeckel, et al., “A survey of stent designs”, Min Invas Ther & Allied Technol. 2002: 11(4) 137-147, the contents of which are incorporated herein by reference.

Referring to FIG. 10, a presently preferred embodiment of the invention includes an expandable portion 160 comprising a layer of a braided mesh of expandable material 150. A presently preferred expandable material for the braided mesh expandable material 150 is nitinol. The braided mesh layer of expandable material 150 can be embedded within the wall 165 of the expandable portion 160 of catheter 100. When the braided mesh of expandable material 150 is expanded, (e.g. by exposure to an expansion temperature), the expandable portion 160 of the tube 101 expands radially to close the opening 300 between catheter 100 and the non-conformable tissue 200, and conforms to the surrounding non-conformable tissue 200 to provide a substantially fluid-tight seal 301 between the exterior of catheter 100 and the non-conformable tissue 200. Leakage of CSF during surgery with catheter 100 is reduced and/or substantially prevented by the seal 301.

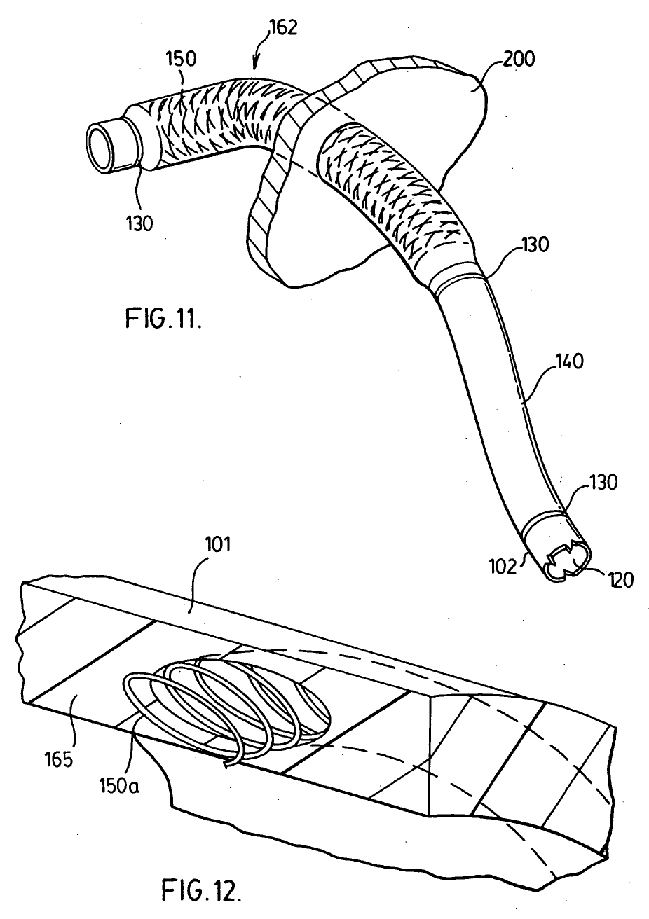

Referring to FIG. 11, the layer of braided mesh expandable material 150 provides the expandable portion 160 of the tube 101 of catheter 100 with a rigidity, stability and strength such that tube 101 is substantially non-compressible and non-collapsable along its length. This expandable portion 160 of the catheter 100, although flexible and capable of being pushed and bent into and through a patient's tissue 200, is substantially resistant to kinking such that the central lumen 120 of tube 101 remains substantially open when bent. In addition, the layer of braided expandable material 150 provides torsional stability while at the same time minimizing (or at least reducing) the wall thickness of catheter 100, and thus maximizing (or at least increasing) the diameter of central lumen 120.

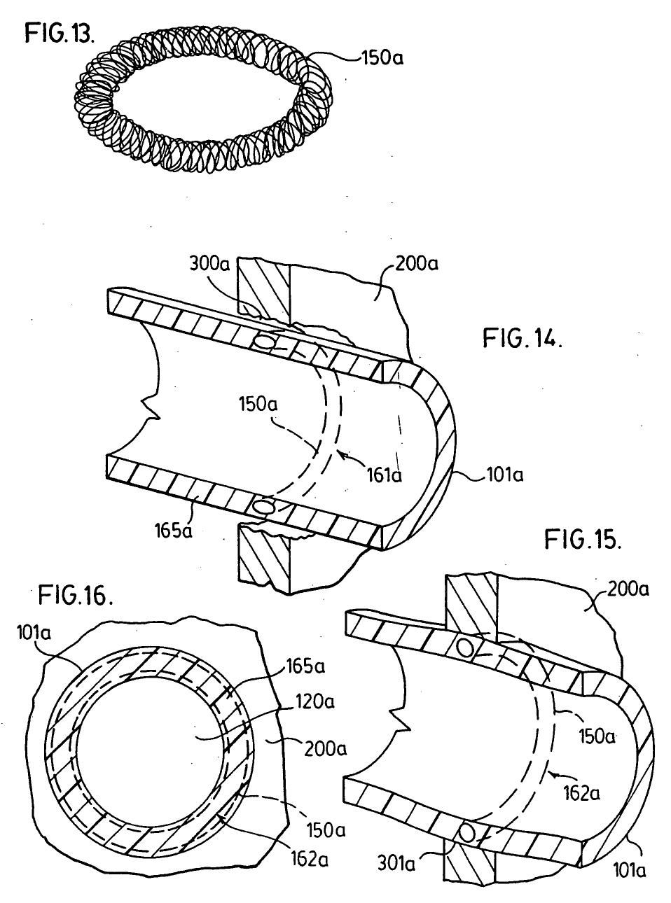

Referring to FIGS. 12-16, another embodiment of the invention includes an expandable portion 160a of catheter 100a comprising expandable material 150a in a toroidal configuration. The catheter 100a of this embodiment, other than having its expandable material 150a in a toroidal configuration, is substantially the same as other embodiments, but is identified by reference numbers followed by the suffix “a”. The toroidal expandable material 150a can be embedded in the wall 165a of the tube portion 101a of catheter 100a. A radial force is exerted outwards as the toroidal expandable material 150a expands radially to the expanded state 162a to allow the expandable portion 160a to conform to the surrounding non-conformable tissue 200a to provide a substantially fluid-tight seal 301a between the exterior of catheter 100a and the non-conformable tissue 200a. When in an expanded state 162a, the expandable portion 160a closes the opening 300a between catheter 100a and the non-conformable tissue 200a. Leakage of CSF during surgery with catheter 100a is reduced and/or substantially prevented by this seal 301a. The toroidal expandable material 150a can be in a variety of configurations, such as a helix of one or more wires, or a solid ring-shaped structure.

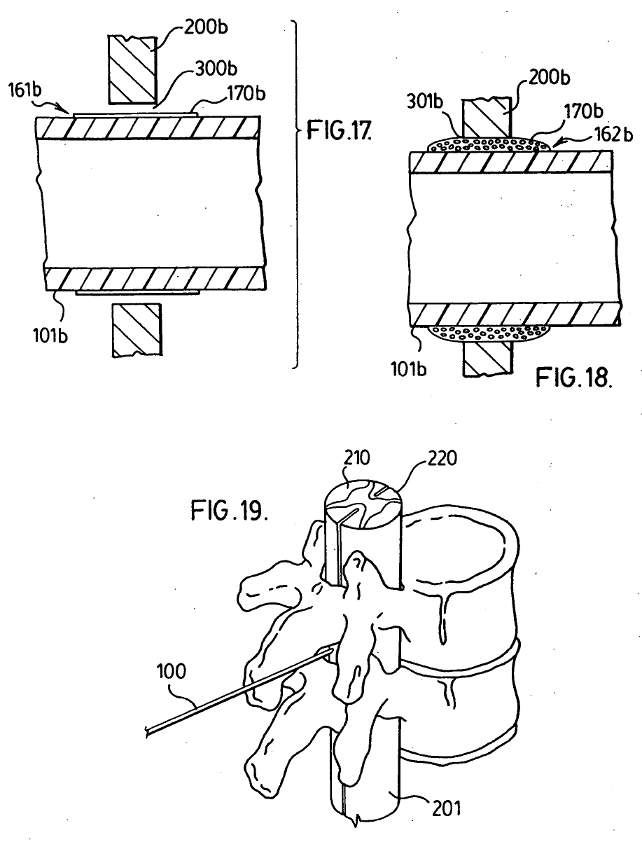

Referring to FIGS. 17 and 18, another embodiment of the invention includes tube 101b of catheter 100b with the expandable portion 160b coated with an expandable coating 170b on its external surface. The catheter 100b of this embodiment, other than having an expandable coating 170b, is substantially the same as other embodiments, but is identified by reference numbers followed by the suffix “b”. When the expandable coating 170b is expanded, (e.g. by exposure to an expansion temperature), it conforms to the surrounding non-conformable tissue 200b to provide a substantially fluid-tight seal 301b between the exterior of catheter 100b and the non-conformable tissue 200b. When in an expanded state 162b, the expandable coating 170b closes the opening 300b between catheter 100b and the non-conformable tissue 200b. Leakage of CSF during surgery with catheter 100b is reduced and/or substantially prevented by this seal 301b.

The expandable coating 170b remains in a non-expanded state 161b at temperatures below a “non-expansion temperature”, and expands to an expanded state 162b at an “expansion temperature” that is above the non-expansion temperature. The Non-expansion temperature is presently preferred to be from about 20° C. to about 30° C., and more presently preferred to be about 25° C. The expansion temperature is presently preferred to be from about 30° C. to about 45° C., and more presently preferred to be about 37.5° C. The expansion temperature is presently preferred to approximate a patient's body temperature. Expansion of expandable coating 170b can also occur with exposure to CSF fluid or other bodily fluid.

It is presently preferred that the expandable coating 170b is made of a material that is macroporous, hydrophilic, biocompatible, adhesion-resistant and infection-resistant. A presently preferred material for the expandable coating 170b is a hydrogel polymer. Examples of a hydrogel polymer are described in U.S. Pat. No. 5,750,585 to Park et. al.; Kallmes D F, Fujiwara N H. New expandable hydrogel-platinum coil hybrid device for aneurysm embolization. AJNR Am J Neuroradiol. 2002 October;23(9):1580-8.] the disclosures of which are incorporated herein by reference. The material for the expandable coating 170b is presently preferred to have a void ratio of at least about 90%, and its hydrophilic properties to be such that it has a water content of at least about 90% when fully hydrated. In a presently preferred embodiment, the expandable coating 170b in the non-expanded state 161b has an initial thickness of not more than about 0.5 mm prior to expansion in situ, with a thickness in the expanded state 162b of at least about 3 mm. The expandable coating 170b is expandable to many times from its initial non-expanded volume, primarily by the hydrophilic absorption of water molecules from an aqueous solution (e.g., resident blood plasma and/or injected saline solution), and secondarily by the filling of its pores with blood. It is presently preferred that the expandable coating 170b expands to volume of at least about 25 times from its non-expanded volume, more presently preferred to be at least about 70 times from its non-expanded volume, and still more presently preferred to be about 100 times from its non-expanded volume. Also, the expandable coating 170b can be coated with a water-soluble coating, such as a starch, to provide a time delayed expansion. Another alternative is to coat the expandable coating 170b with a temperature-sensitive coating that disintegrates in response to normal human body temperature. (See, e.g., U.S. Pat. No. 5,120,349—Stewart et al. and U.S. Pat. No. 5,129,180—Stewart.)

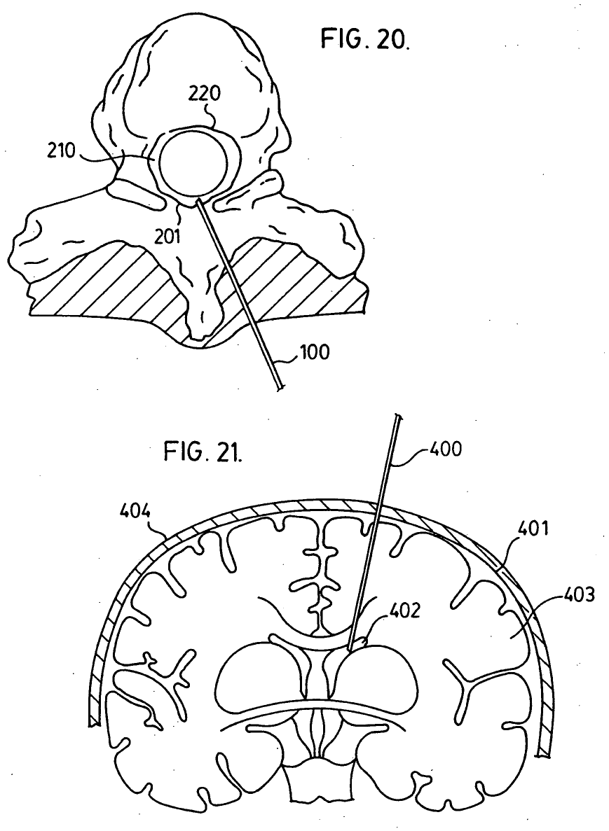

Referring to FIGS. 19-21, although the present invention has been described herein in connection with catheter 100 being suitable for insertion through a patient's dura mater 201 to provide access to a patient's cerebral spinal fluid (“CSF”) in the cerebrospinal canal 210 of the spinal cord 220, it is contemplated that the present invention can also be practiced in connection with a catheter 400 for insertion into other non-conformable tissues within a patient's body, such as for providing access to the CSF in the ventricles 402 through a patient's brain 403 (FIG. 21). As will occur to those of skill in the art, examples of non-conformable tissue include the dura mater 201 that covers the spinal cord 220, the dura mater 401 that covers the brain 403, and the brain 403. Non-conformable tissue includes any tissue within a patient's body that is substantially non-elastic and does not conform to objects inserted through it.

While only specific combinations of the various features and components of the present invention have been discussed herein, it will be apparent to those of skill in the art that desired subsets of the disclosed features and components and/or alternative combinations of these features and components can be utilized, as desired. For example, the embodiments discussed herein can be combined to further the expansion of the catheter to conform to surrounding non-conformable tissue. For example, it is contemplated that expandable coating 170b can be used to coat the external surface of expandable portion 160 comprising expandable material 150, and similarly, to coat the external surface of expandable portion 160a comprising expandable material 150a.

The above-described embodiments of the invention are intended to be examples of the present invention and alterations and modifications can be effected thereto, by those of skill in the art, without departing from the scope of the invention which is defined solely by the claims appended hereto.

I claim:

1. A catheter for use in a non-conformable tissue, said catheter comprising a tube for insertion into the non-conformable tissue, said tube further comprising a first portion and a second portion, said first portion being expandable from a non-expanded state, which permits insertion of the catheter into the non-conformable tissue, to an expanded state, which is conformable to the non-conformable tissue such that the first portion of the tube in the expanded state provides a seal between an exterior of said catheter and the non-conformable tissue.

2. The catheter of claim 1 wherein the first portion of the tube is expanded by an increase in temperature.

3. The catheter of claim 2 wherein the first portion of the tube expands to the expanded state at temperatures above about 30° C.

4. The catheter of claim 3 wherein the first portion of the tube expands to the expanded state at a temperature between about 30° C. and about 45° C.

5. The catheter of claim 4 wherein the first portion of the tube expands to the expanded state at a temperature of about 37.5° C.

6. The catheter of claim 1 wherein the first portion of the tube remains in the non-expanded state at temperatures below about 30° C.

7. The catheter of claim 6 wherein the first portion of the tube remains in the non-expanded state at temperatures below about 25° C.

8. The catheter of claim 1 wherein the first portion of the tube comprises nitinol.

9. The catheter of claim 8 wherein the nitinol is embedded in an outside wall of the first portion of the tube in a configuration selected from the group consisting of a braided mesh layer of one or more wires, a cable of one or more wires, a helix of one or more wires, a sheet-like expanse, a toroid, a coil, a helical spiral, a ring, a sequence of rings, a closed cell, and an open cell.

10. The catheter of claim 8 wherein the nitinol provides a radial force that substantially prevents the first portion of the tube from collapsing when bent.

11. The catheter of claim 1 wherein the first portion of the tube is between about 5 cm and about 15 cm in length.

12. The catheter of claim 11 wherein the first portion of the tube is about 10 cm in length.

13. The catheter of claim 1 wherein the second portion of the tube is flexible.

14. The catheter of claim 1 wherein the second portion of the tube is between about 5 cm and about 15 cm in length.

15. The catheter of claim 14 wherein the second portion of the tube is about 10 cm in length.

16. The catheter of claim 1 wherein the tube further comprises at least one radiopaque marker.

17. The catheter of claim 16 wherein the at least one radio-opaque marker is located at at least one end of the first portion, and at the distal end of the second portion.

18. The catheter of claim 16 wherein the radiopaque marker includes a material selected from the group consisting of barium sulfate, gold, iodine, ionic and non ionic iodinated compounds, ethiodol, and lipiodol, tungsten, tantalum, gadolinium nitinol, silver, and combinations thereof.

19. The catheter of claim 1 wherein the first portion of the tube further comprises an expandable coating surrounding the tube, the expandable coating being expandable from a non-expanded state, which permits insertion of the catheter into the non-conformable tissue, to an expanded state, which is conformable to the non-conformable tissue such that the expandable coating in the expanded state provides a seal between an exterior of the catheter and the non-conformable tissue.

20. The catheter of claim 19 wherein the expandable coating is expanded by an increase in temperature.

21. The catheter of claim 20 wherein the expandable coating expands to the expanded state at temperatures above about 30° C.

22. The catheter of claim 21 wherein the expandable coating expands to the expanded state at a temperature between about 30° C. and about 45° C.

23. The catheter of claim 22 wherein the expandable coating expands to the expanded state at a temperature of about 37.5° C.

24. The catheter of claim 19 wherein the expandable coating remains in the non-expanded state at temperatures below about 30° C.

25. The catheter of claim 24 wherein the expandable coating remains in the non-expanded state at a temperature below about 25° C.

26. The catheter of claim 19 wherein the expandable coating comprises a hydrogel polymer.

27. The catheter of claim 1 wherein the tube is tapered.

28. The catheter of claim 1 wherein at least a portion of the catheter has an antibiotic coating.

29. The catheter of claim 1 wherein at least a portion of the catheter has an adhesion resistant coating.

30. The catheter of claim 1 wherein the non-conformable tissue is a dura mater.

31. The catheter of claim 1 wherein the non-conformable tissue is a brain.

32. A catheter for use in a non-conformable tissue, comprising
a tube for insertion into the non-conformable tissue; and
an expandable coating surrounding the tube, the expandable coating being expandable from a non-expanded state, which permits insertion of the catheter into the non-conformable tissue, to an expanded state, which is conformable to the non-conformable tissue such that the expandable coating in the expanded state provides a seal between an exterior of the catheter and the non-conformable tissue.

33. The catheter of claim 32 wherein the expandable coating is expanded by an increase in temperature.

34. The catheter of claim 32 wherein the expandable coating comprises a hydrogel polymer.

35. The catheter of claim 32 wherein the tube further comprises a first portion and a second portion, said first portion being expandable from a non-expanded state, which permits insertion of the catheter into the non-conformable tissue, to an expanded state, which is conformable to the non-conformable tissue such that the first portion of the tube in the expanded state provides a seal between an exterior of said catheter and the non-conformable tissue.

36. The catheter of claim 35 wherein the first portion of the tube is expanded by an increase in temperature.

37. The catheter of claim 35 wherein the first portion of the tube comprises nitinol.
